(19) United States
(12) Patent Application Publication
(10) Pub. No.: US 2010/0291158 A1
(43) Pub. Date: Nov. 18, 2010

(54) SUSTAINED RELEASE ORAL COMPOSITION OF AN ANTIPSYCHOTIC AGENT

(75) Inventors: Deepak Ashok Kumar Murpani, New Delhi (IN); Pandora Alexaki, Athens (GR)

Correspondence Address: SUGHRUE MION, PLLC, 2100 PENNSYLVANIA AVENUE, N.W., SUITE 800, WASHINGTON, DC, 20037, US

(73) Assignee: GENEPHARM TRADING, Mumbai (IN)

(21) Appl. No.: 12/729,674

(22) Filed: Mar. 23, 2010

(30) Foreign Application Priority Data
Mar. 23, 2009 (IN) 670/MUM/2009

(51) Int. Cl. A61K 31/554; A61P 25/18; A61K 9/00

(52) U.S. Cl. 424/400; 514/211.13

(57) ABSTRACT

A sustained release oral composition of an antipsychotic agent comprising antipsychotic agent, λ-carrageenan and one or more pharmaceutically acceptable excipients; wherein the antipsychotic agent is 2-[2-(4-dibenzo{b,f}[1,4-thiazepin-11yl-1-piperazinyl)ethoxy ethanol] or its pharmaceutically acceptable salt.

The present invention relates to a sustained release oral pharmaceutical composition of an antipsychotic agent, -[2-(4-dibenzo{b,f}[1,4-thiazepin-11yl-1-piperazinyl)ethoxy ethanol] or quetiapine (INN name) and its pharmaceutically acceptable salt. Antipsychotic agents are normally used to treat psychotropic disorders and other mental/emotional conditions including schizophrenia and acute manic episodes associated with bipolar I disorder.

### BACKGROUND OF THE INVENTION

U.S. Pat. No. 4,879,288 (hereinafter “the '288 patent”) discloses the compound 11-[4-[2-(2-hydroxyethoxy)ethyl]-1-piperazinyl}dibenzo[b,f][1,4]thiazepine as a novel antipsychotic drug of the dibenzothiazepine class suitable for various psychotropic disorders and having less side effects. The '288 patent exemplifies immediate release tablets of 11-[4-[2-(2-hydroxyethoxy)ethyl]-1-piperazinyl}dibenzo[b,f][1,4]thiazepine comprising lactose, pregelatinized starch, maize starch, stearic acid, polyvinylpyrrolidone, and magnesium stearate.

The use of a sustained release oral dosage form of phenothiazines derivatives is a desired approach in the treatment of psychotropic disorders. A sustained release formulation
(a) avoids frequent administration of the drug while maintaining uniform and constant release rate of the active pharmaceutical ingredient over an extended period of time; and
(b) maintains effective plasma drug concentration and controlled release rate of medicament after a solid oral drug administration.

PCT Publication No. WO 97/45124 discloses a sustained release formulation comprising a gelling agent and quetiapine or a pharmaceutically acceptable salt thereof, together with one or more pharmaceutically acceptable excipients. The gelling agent is hydroxypropyl methyl cellulose and the pharmaceutically acceptable excipient is selected from the group consisting of macrocrystalline cellulose, lactose, magnesium stearate, sodium citrate and povidone. Alternate formulations with non-gelling agents may provide sustained release at a steady rate.

PCT Publication No. WO 01/21179 discloses a granule formulation comprising quetiapine or a pharmaceutically acceptable salt thereof and a freely or very water-soluble binder. The granules are prepared by using fluid bed technology. The granules are dissolved or suspended in an aqueous medium and then administered to patients with central nervous system disorders.

PCT Publication No. WO 03/039516 discloses a method for improving the dissolution of a poorly dispersible medicament such as quetiapine, which comprises mixing the poorly dispersible medicament with a floating agent and/or a surfactant and granulating the mixture.

PCT Publication No. WO 2005/041935 discloses a sustained release solid dosage formulation comprising a matrix, wherein the matrix comprises a therapeutically effective amount of quetiapine or a pharmaceutically acceptable salt thereof, and a wax material. These formulations may not provide sustained release at a steady rate and also may provide incomplete drug release due to hydrophobic nature of wax.

We have now surprisingly found that a sustained release dissolution profile of quetiapine is obtained by using lambda carrageenan in the dosage form with an active pharmaceutical ingredient, such as quetiapine or its pharmaceutically acceptable salt.

The object of the present invention is to provide a sustained release oral composition of an antipsychotic agent, such as 2-[2-(4-dibenzo{b,f}[1,4-thiazepin-11yl-1-piperazinyl)ethoxy ethanol] or its pharmaceutically acceptable salt with the use of lambda carrageenan.

### SUMMARY OF THE INVENTION

Provided herein is a sustained release oral composition of an antipsychotic agent comprising an antipsychotic agent, λ-carrageenan and one or more pharmaceutically acceptable excipients. The antipsychotic agent may be 2-[2-(4-dibenzo{b,f}[1,4-thiazepin-11yl-1-piperazinyl)ethoxy ethanol] or its pharmaceutically acceptable salt.

Also provided herein is a process for the preparation of a sustained release oral composition of an antipsychotic agent comprising
(i) mixing an antipsychotic agent with λ-carrageenan and one or more pharmaceutically acceptable excipients;
(ii) compacting or granulating the mixture of (i) followed by milling;
(iii) drying and optionally compressing;
   wherein the antipsychotic agent may be 2-[2-(4-dibenzo{b,f}[1,4-thiazepin-11yl-1-piperazinyl)ethoxy ethanol] or its pharmaceutically acceptable salt.

### DETAILED DESCRIPTION OF THE INVENTION

One embodiment of the present invention is a sustained release oral composition of 2-[2-(4-dibenzo{b,f}[1,4-thiazepin-11yl-1-piperazinyl)ethoxy ethanol] or its pharmaceutically acceptable salt with lambda carrageenan and one or more pharmaceutically acceptable excipients.

The use of lambda-carrageenan reportedly provides distinct advantages such as
(a) zero order or steady release of 2-[2-(4-dibenzo{b,f}[1,4-thiazepin-11yl-1-piperazinyl)ethoxy ethanol]
(b) provides a steady release of 2-[2-(4-dibenzo{b,f}[1,4-thiazepin-11yl-1-piperazinyl)ethoxy ethanol] in most organic solvents as lambda carrageenan is insoluble in them. Therefore by consumption of alcohol, the pharmacokinetic profile is not affected.
(c) As it is pH independent it provides a constant rate of release in the GIT.

The USPNF 23 describes carrageenan as a hydrocolloid obtained by extraction with water or an aqueous alkali from some members of the class Rhodophyceae (red seaweed). It consists chiefly of potassium, sodium, calcium magnesium and ammonium sulfate esters of galactose and 3,6-anhydrogalactose copolymers. These hexoses are alternatively linked at the α-1,3 and β-1,4 sites in the polymer.

The carrageenans are divided into three families according to the position of sulfate groups and the presence of anhydrogalactose.

λ-Carrageenan (lambda-carrageenan) is a nongelling polymer containing about 35% ester sulfate by weight and no 3,6 anhydrogalactose.

ι-Carrageenan (iota-carrageenan) is a gelling polymer containing about 32% ester sulfate by weight and approximately 30% 3,6 anhydrogalactose.

κ-Carrageenan (kappa-carrageenan) is a strongly gelling polymer which has a helical tertiary structure that allows gelling. It contains 25% ester sulfate by weight and approximately 34% 3,6 anhydrogalactose.

Among the three carrageenans, we have found that the lambda (λ-) carrageenan is the only nongelling polymer.

λ-Carrageenan, when formulated with 2-[2-(4-dibenzo{b,f}[1,4-thiazepin-11yl-1-piperazinyl)ethoxy ethanol] or its pharmaceutically acceptable salt and optionally other pharmaceutical excipient(s), provides sustained or extended release of 2-[2-(4-dibenzo{b,f}[1,4-thiazepin-11yl-1-piperazinyl)ethoxy ethanol].

The weight ratio of 2-[2-(4-dibenzo{b,f}[1,4-thiazepin-11yl-1-piperazinyl)ethoxy ethanol] or its pharmaceutically acceptable salt to λ-carrageenan may range from 1:0.1 to 1:10.

The sustained release oral composition of the present invention comprises one or more pharmaceutical excipient(s). The pharmaceutical excipient used in the oral composition of the present invention must be compatible with 2-[2-(4-dibenzo{b,f}[1,4-thiazepin-11yl-1-piperazinyl)ethoxy ethanol] or its pharmaceutically acceptable salt. The pharmaceutical excipient may be selected from diluents, binders, lubricants, disintegrants, flavoring agents, coloring agents, stabilizers, surfactants, glidants, plasticizers, preservatives and sweeteners.

Diluents may be selected from calcium carbonate, calcium phosphate dibasic, calcium phosphate tribasic, calcium sulfate, microcrystalline cellulose, microcrystalline silicified cellulose, powdered cellulose, dextrates, dextrose, fructose, lactitol, lactose anhydrous, lactose monohydrate, lactose dihydrate, lactose trihydrate, mannitol, sorbitol, starch, pregelatinized starch, sucrose, talc, xylitol, maltose, maltodextrin, maltitol.

Binders may be selected from acacia, alginic acid, carbomer, carboxymethylcellulose calcium, carbomethylcellulose sodium, microcrystalline cellulose, powdered cellulose, ethyl cellulose, gelatin liquid glucose, guar gum, hydroxyethyl cellulose, hydroxypropyl cellulose, hydroxypropylmethyl cellulose, maltodextrin, methylcellulose, polydextrose, polyethtylene oxide, povidone, sodium alginate, starch paste, pregelatinized starch, sucrose, tragacanth, low-substituted hydroxypropyl cellulose, glucose, sorbitol. Suitable fillers are preferably selected from at least one of starch derivatives, such as corn starch, potato starch or rice starch. Polysaccharides such as dextrins, maltodextrins, dextrates, microcrystalline cellulose, powdered cellulose, mixture of microcrystalline cellulose and guar gum, coprocessed blends of microcrystalline cellulose; and polyhydric alcohols, such as xylitol and sorbitol.

Disintegrants may be selected from alginic acid, carbon dioxide, carboxymethylcellulose calcium, carboxymethylcellulose sodium, microcrystalline cellulose, powdered cellulose, croscarmelose sodium, crospovidone, sodium docusate, gaur gum, hydroxypropyl cellulose, methylcellulose, polacrilin potassium, poloxamer, povidone, sodium alginate, sodium glycine carbonate, sodium laulyl sulfate, sodium starch glycolate, starch, pregelatinized starch, low-substituted hydroxypropyl cellulose.

Glidants may be selected from calcium silicate, powdered cellulose, starch, talc, colloidal silicon dioxide.

Lubricants may be selected from magnesium stearate, stearic acid, sodium stearyl fumarate, magnesium lauryl sulphate, talc, polyethylene glycol, and glyceryl behenate.

Suitable sweeteners may be selected from sugars such as sucrose, lactose and glucose; cyclamate and salts thereof; saccharin and salts thereof; and aspartame.

Flavouring agents may be selected from natural or synthetic flavours such as strawberry flavour, wild cherry flavour, green apple flavour, spearmint flavour and peppermint flavour.

Another embodiment of the present invention is a sustained release oral composition of 2-[2-(4-dibenzo{b,f}[1,4-thiazepin-11yl-1-piperazinyl)ethoxy ethanol] or its pharmaceutically acceptable salt with lambda carrageenan and one or more pharmaceutically acceptable excipients wherein the dissolution profile of the oral composition shows that it releases from 10 to 40% of its 2-[2-(4-dibenzo{b,f}[1,4-thiazepin-11yl-1-piperazinyl)ethoxy ethanol] in the first 2 hours of administration and more than 60% of its 2-[2-(4-dibenzo{b,f}[1,4-thiazepin-11yl-1-piperazinyl)ethoxy ethanol] over the first 12 hours after administration. It is more preferable that the dissolution profile shows the release of from 60% to 90% of the 2-[2-(4-dibenzo{b,f}[1,4-thiazepin-11yl-1-piperazinyl)ethoxy ethanol] over the first 12 hours of administration. It is also more preferable that the dissolution profile shows the release of at least 70% of the 2-[2-(4-dibenzo{b,f}[1,4-thiazepin-11yl-1-piperazinyl)ethoxy ethanol] in the first 16 hours after administration.

Yet another embodiment of the present invention is a process for the preparation of sustained release oral composition of 2-[2-(4-dibenzo{b,f}[1,4-thiazepin-11yl-1-piperazinyl)ethoxy ethanol] or its pharmaceutically acceptable salt comprising 2-[2-(4-dibenzo{b,f}[1,4-thiazepin-11yl-piperazinyl)ethoxy ethanol] or its pharmaceutically acceptable salt, lambda carrageenan and one or more pharmaceutical excipient(s). In this embodiment, 2-[2-(4-dibenzo{b,f}[1,4-thiazepin-11yl-piperazinyl)ethoxy ethanol], lambda carrageenan and one or more pharmaceutical excipient(s) are mixed, compacted or granulated, milled, dried and optionally compressed. A coating may be applied to the dosage form.

The dosage form of the present invention may be prepared by using conventional techniques employed in the art for mixing, compaction, granulation, milling, drying and compressing.

The oral composition may be selected from sprinkle granules or powder for reconstitution in a suspension, tablet, soluble tablet, rapidly disintegrating tablet, orally disintegrating tablet, rapidly disintegrating film, orally disintegrating powder for capsules, suspension or sachets, effervescent tablet, a chewable tablet, water dispersible tablet, orodispersible tablet, a chewing gum and suspension.

If the dosage form is coated, it may be coated with a polymeric non-functional coating wherein the polymer(s) in the coating are selected from hydroxypropyl methyl cellulose, hydroxypropyl methyl cellulose phthalate, polyvinyl acetate phthalate, cellulose acetate phthalate, hydroxypropyl methylcellulose, hydroxyethyl cellulose, hydroxypropyl cellulose, methyl hydroxyethylcellulose, sodium carboxy methylcellulose, polyvinylpyrrolidone, polyvinylalcohol, methyl cellulose, ethyl cellulose, polyvinylacetate, dimethylaminoethyl methacrylate and the like.

### EXAMPLES

The oral composition of the present invention was subjected to a dissolution method (0.1N hydrochloric acid 750 ml, paddle 50 rpm. After 2 hours, 250 ml of phosphate buffer was added to obtain a pH 6.2).

The following examples illustrate preferred embodiments in accordance with the present invention but should not be considered to limit the scope of the present invention in any manner.

Table 1 illustrates the formulations of Examples 1-7. Examples 1-4 contain a weight ratios of 2-[2-(4-dibenzo{b,f}[1,4-thiazepin-11yl-1-piperazinyl)ethoxy ethanol] to λ-carrageenan of 1:1.1. Examples 5 and 6 contain a weight ratios of 2-[2-(4-dibenzo{b,f}[1,4-thiazepin-11yl-1-piperazinyl)ethoxy ethanol] to λ-carrageenan of 1:0.9. Example 7 contains a weight ratio of 2-[2-(4-dibenzo{b,f}[1,4-thiazepin-11yl-1-piperazinyl)ethoxy ethanol] to λ-carrageenan of 1:0.7.

With respect to the manner in which each of Examples 1-7 was formulated, as noted in Table 1, Examples 1 and 2 were created by compacting and sizing through 24 mesh. Examples 3-5 were created using a wet granulation process with purified water. Examples 6 and 7 were created using a wet granulation process with a PVP binder solution.

TABLE 1

| | Examples | | | | | | |
|---|---|---|---|---|---|---|---|
| | 1 | 2 | 3 | 4 | 5 | 6 | 7 |
| Ingredients | mg/tab | mg/tab | mg/tab | mg/tab | mg/tab | mg/tab | mg/tab |
| Quetiapine | 230.5 | 230.5 | 230.5 | 230.5 | 230.5 | 230.5 | 230.5 |
| hemifumarate | | | | | | | |
| Viscarin 209 (Lambda- | 250 | 250 | 250 | 250 | 200 | 200 | 150 |
| Carrageenan) | | | | | | | |
| Microcrystalline | 64.5 | — | 64.5 | 113.5 | 113.5 | 99 | 99 |
| cellulose | | | | | | | |
| Lactose SD | — | 64.5 | — | — | — | — | — |
| Glyceryl behenate | — | — | — | — | — | — | 50 |
| Povidone K30 | — | — | — | — | — | 15 | 15 |
| Mg Stearate | 5 | 5 | 5 | 6 | 6 | 5.5 | 5.5 |
| Total weight | 550 | 550 | 550 | 600 | 550 | 550 | 550 |
| Process for the | Compaction | Compaction | Wet | Wet | Wet | Wet | Wet |
| preparation of | and sizing | and sizing | granulation | granulation | granulation | granulation | granulation |
| granules before | through 24 | through 24 | with purified | with purified | with purified | with PVP | with PVP |
| compression into | mesh | mesh | water | water | water | binder solution | binder solution |
| tablets | | | | | | | |

TABLE 2
Dissolution profiles of oral compositions of Examples 3-6

| Dissolution | Exam- | Exam- | Exam- | | | Exam- |
|---|---|---|---|---|---|---|
| profile | ple 3 | ple 4 | ple 5 | | | ple 6 |
| TIME(h) | 50 rpm | 50 rpm | 50 rpm | 50 rpm | 100 rpm | 100 rpm |
| 1 | 10.6 | 9.2 | 14.0 | 14.5 | 17.4 | 15.3 |
| 2 | 19.4 | 17.4 | 26.5 | 27.6 | 31.3 | 27.1 |
| 4 | 31.2 | 30.8 | 39.0 | 39.8 | 47.5 | 42.1 |
| 6 | 41.2 | 41.7 | 49.0 | 48.6 | 60.4 | 54.9 |
| 8 | 50.6 | 51.1 | 56.7 | 56.7 | 71.4 | 66.0 |
| 12 | 67.3 | 65.4 | 69.9 | 70.6 | 89.9 | 84.1 |
| 16 | 78.2 | 77.0 | 80.7 | 80.6 | 99.4 | 94.0 |

Table 2 illustrates the dissolution profiles of Examples 3-5, as tested at the rpms shown in the Table. The table reports the percentage of quetiapine hemifumarate released over time from the Examples. As can be seen from the Table, each of Examples 3-6 releases over about 60% of its quetiapine hemifumarate over the first 12 hours after administration, and more specifically the Examples release between about 60 and about 90% of their quetiapine hemifumarate in the first 12 hours after administration. It is noted that the Examples in the Table show a release of over about 70% of quetiapine hemifumarate in the first 16 hours after administration.

TABLE 3
Dissolution profile of the inventors' SEROQUEL tablets

| | |
|---|---|
| SEROQUEL | SEROQUEL |
| 200 mg NN0049 | 200 mg FL076 |
| (CANADA) | (GREECE) |

| Time (hrs) | 50 rpm | 50 rpm | 100 rpm |
|---|---|---|---|
| 1 | 18.7 | 17.8 | 21.7 |
| 2 | 33.0 | 30.8 | 38.0 |
| 4 | 39.8 | 38.7 | 49.2 |
| 6 | 46.5 | 43.5 | 56.5 |
| 8 | 53.9 | 49.3 | 65.3 |
| 12 | 66.8 | 60.9 | 79.9 |
| 16 | 82.0 | 70.3 | 89.9 |

Table 3 illustrates the dissolution profiles of some of the inventors' SEROQUEL 200 mg tablets. As can be seen from the Table, the SEROQUEL 200 mg tablets in the Table release over about 30% of their quetiapine hemifumarate in the first 2 hours after administration; between about 60% and about 80% of their quetiapine hemifumarate in the first 12 hours after administration; and between about 70% and about 90% of their quetiapine hemifumarate in the first 16 hours after administration.

We claim:

1. A sustained release oral composition of an antipsychotic agent comprising antipsychotic agent, λ-carrageenan and one or more pharmaceutically acceptable excipients; wherein the antipsychotic agent is 2-[2-(4-dibenzo{b,f}[1,4-thiazepin-11yl-1-piperazinyl)ethoxy ethanol] or its pharmaceutically acceptable salt.

2. A sustained release oral composition as claimed in claim 1 wherein the oral composition is selected from sprinkle granules or powder for reconstitution in a suspension, tablet, soluble tablet, rapidly disintegrating tablet, orally disintegrating tablet, rapidly disintegrating film, orally disintegrating powder for capsules, suspension or sachets, effervescent tablet, a chewable tablet, water dispersible tablet, orodispersible tablet, a chewing gum and suspension.

3. A sustained release oral composition as claimed in any one of claim 1 or 2 wherein the pharmaceutical excipient is selected from diluents, binders, lubricants, disintegrants, flavoring agents, coloring agents, stabilizers, surfactants, glidants, plasticizers, preservatives and sweeteners.

4. A sustained release oral composition as claimed in claim 3 wherein the pharmaceutical excipient is selected from lactose, povidone, glyceryl behenate, microcrystalline cellulose and magnesium stearate.

5. A sustained release oral composition as claimed in claim 4 wherein the pharmaceutical excipient is povidone.

6. A sustained release oral composition as claimed in claim 1 wherein the ratio of 2-[2-(4-dibenzo{b,f}[1,4-thiazepin-11yl-1-piperazinyl)ethoxy ethanol] or its pharmaceutically acceptable salt to λ-carrageenan is 1:0.1 to 1:10.

7. A sustained release oral composition as claimed in claim 1 further comprising a coating.

8. A sustained release oral composition as claimed in claim 1 wherein the dissolution profile of the oral composition releases greater than 60% in more than 12 hrs of 2-[2-(4-dibenzo{b,f}[1,4-thiazepin-11yl-1-piperazinyl)ethoxy ethanol].

9. A sustained release oral composition as claimed in claim 1 wherein the dissolution profile of the oral composition releases less than 17% in one hour of 2-[2-(4-dibenzo{b,f}[1,4-thiazepin-11yl-1-piperazinyl)ethoxy ethanol].

10. A sustained release oral composition as claimed in claim 1 wherein the dissolution profile of the oral composition releases less than 32% in 2 hrs of 2-[2-(4-dibenzo {b,f}[1,4-thiazepin-11yl-1-piperazinyl)ethoxy ethanol].

11. A sustained release oral composition as claimed in claim 1 wherein the dissolution profile of the oral composition releases less than 48% in 4 hrs of 2-[2-(4-dibenzo {b,f}[1,4-thiazepin-11yl-1-piperazinyl)ethoxy ethanol].

12. A sustained release oral composition as claimed in claim 1 wherein the dissolution profile of the oral composition releases less than 60% in 6 hrs of 2-[2-(4-dibenzo {b,f}[1,4-thiazepin-11yl-1-piperazinyl)ethoxy ethanol].

13. A sustained release oral composition as claimed in claim 1 wherein the dissolution profile of the oral composition releases less than 72% in 8 hrs of 2-[2-(4-dibenzo {b,f}[1,4-thiazepin-11yl-1-piperazinyl)ethoxy ethanol].

14. A sustained release oral composition as claimed in claim 1 wherein the dissolution profile of the oral composition releases less than 90% in 12 hrs of 2-[2-(4-dibenzo {b,f}[1,4-thiazepin-11yl-1-piperazinyl)ethoxy ethanol].

15. A sustained release oral composition as claimed in claim 14 wherein the dissolution profile of the oral composition releases more than 65% in 12 hrs of 2-[2-(4-dibenzo{b,f}[1,4-thiazepin-11yl-1-piperazinyl)ethoxy ethanol].

16. A sustained release oral composition as claimed in claim 1 wherein the dissolution profile of the oral composition releases more than 77% in 16 hrs of 2-[2-(4-dibenzo{b,f}[1,4-thiazepin-11yl-1-piperazinyl)ethoxy ethanol].

17. A sustained release oral composition as claimed in claim 1 wherein the dissolution profile of the oral composition releases 10 to 40% in 2 hours and greater than 60% in more than 12 hrs of 2-[2-(4-dibenzo{b,f}[1,4-thiazepin-11yl-1-piperazinyl)ethoxy ethanol].

18. A sustained release oral composition as claimed in claim 1 wherein the dissolution profile of the oral composition shows a release of 15 to 32% in 2 hrs and 65% to 90% in 12 hrs of 2-[2-(4-dibenzo{b,f}[1,4-thiazepin-11yl-1-piperazinyl)ethoxy ethanol].

19. A sustained release oral composition of an antipsychotic agent comprising antipsychotic agent, λ-carrageenan, povidone and one or more pharmaceutically acceptable excipients; wherein the antipsychotic agent is 2-[2-(4-dibenzo{b,f}[1,4-thiazepin-11yl-1-piperazinyl)ethoxy ethanol] or its pharmaceutically acceptable salt.

20. A sustained release oral composition of an antipsychotic agent comprising antipsychotic agent, λ-carrageenan and one or more pharmaceutically acceptable excipients; wherein the antipsychotic agent is 2-[2-(4-dibenzo{b,f}[1,4-thiazepin-11yl-1-piperazinyl)ethoxy ethanol] or its pharmaceutically acceptable salt; which is coated.

21. A process for the preparation of a sustained release oral composition of an antipsychotic agent comprising
(i) mixing 2-[2-(4-dibenzo{b,f}[1,4-thiazepin-11yl-1-piperazinyl)ethoxy ethanol] or its pharmaceutically acceptable salt with λ-carrageenan and one or more pharmaceutically acceptable excipients;
(ii) compacting or granulating the mixture of (i) followed by milling;
(iii) drying and optionally compressing;
wherein the antipsychotic agent is 2-[2-(4-dibenzo{b,f}[1,4-thiazepin-11yl-1-piperazinyl)ethoxy ethanol] or its pharmaceutically acceptable salt.

22. A process for the preparation of a sustained release oral composition of an antipsychotic agent comprising
(i) mixing 2-[2-(4-dibenzo{b,f}[1,4-thiazepin-11yl-1-piperazinyl)ethoxy ethanol] or its pharmaceutically acceptable salt with λ-carrageenan, povidone and optionally one or more pharmaceutically acceptable excipients;
(ii) compacting or granulating the mixture of (i) followed by milling;
(iii) drying and optionally compressing;
wherein the antipsychotic agent is 2-[2-(4-dibenzo{b,f}[1,4-thiazepin-11yl-1-piperazinyl)ethoxy ethanol] or its pharmaceutically acceptable salt.

23. A process for the preparation of a sustained release oral composition of an antipsychotic agent comprising
(i) mixing 2-[2-(4-dibenzo{b,f}[1,4-thiazepin-11yl-1-piperazinyl)ethoxy ethanol] or its pharmaceutically acceptable salt with λ-carrageenan and one or more pharmaceutically acceptable excipients;
(ii) compacting or granulating the mixture of (i) followed by milling;
(iii) drying, optionally compressing; and
(iv) coating;
wherein the antipsychotic agent is 2-[2-(4-dibenzo{b,f}[1,4-thiazepin-11yl-1-piperazinyl)ethoxy ethanol] or its pharmaceutically acceptable salt.

24. A process for the preparation of a sustained release oral composition of an antipsychotic agent as claimed in claim 1 comprising
(i) mixing 2-[2-(4-dibenzo{b,f}[1,4-thiazepin-11yl-1-piperazinyl)ethoxy ethanol] or its pharmaceutically acceptable salt with λ-carrageenan, one or more pharmaceutically acceptable excipients and optionally povidone;
(ii) compacting or granulating the mixture of (i) followed by milling;
(iii) drying, optionally compressing; and
(iv) optionally coating;
wherein the antipsychotic agent is 2-[2-(4-dibenzo{b,f}[1,4-thiazepin-11yl-1-piperazinyl)ethoxy ethanol] or its pharmaceutically acceptable salt.
